(19) United States
(12) Patent Application Publication
(10) Pub. No.: US 2019/0112623 A1
(43) Pub. Date: Apr. 18, 2019

(54) A METHOD AND AN APPARATUS FOR AN ENZYMATIC HYDROLYSIS, A LIQUID FRACTION AND A SOLID FRACTION

(71) Applicant: UPM-KYMMENE CORPORATION, Helsinki (FI)

(72) Inventors: Sami Turunen, Lappeenranta (FI); Juha Tamper, Levänen (FI)

(21) Appl. No.: 16/086,145

(22) PCT Filed: Mar. 22, 2017
(86) PCT No.: PCT/FI2017/050201

§ 371 Date: Sep. 18, 2018

(30) Foreign Application Priority Data
Mar. 24, 2016 (FI) 20165250

(51) Int. Cl. C12P 19/02; C12M 1/00

(52) U.S. Cl. CPC C12P 19/02; C12M 45/09; C12M 45/04

(57) ABSTRACT

The invention relates to a method and an apparatus for an enzymatic hydrolysis in which plant based raw material is hydrolysed by means of enzymes. The plant based raw material (1) is fed to the first enzymatic hydrolysis stage (2), the plant based raw material (1) is hydrolysed in at least two enzymatic hydrolysis stages (2,4), a liquid fraction (5a,5b) comprising carbohydrates is separated from a solid fraction (6a,6b) in a solid-liquid separation stage (7a,7b) after each enzymatic hydrolysis stage (2,4), and the solid fraction (6a) is supplied to the next enzymatic hydrolysis stage (4) in which the solid fraction is treated, and the solid fraction (6b) is recovered after the last solid-liquid separation stage (7b). Further, the invention relates to the liquid fraction and the solid fraction and their use.

### FIELD

The invention relates to a method and an apparatus for an enzymatic hydrolysis. Further, the invention relates to a liquid fraction and a solid fraction and their use.

### BACKGROUND

It is known different methods for forming carbohydrates and lignin from different raw materials, such as biomass. Many bio-refinery processes, e.g. hydrolysis, generate lignin and sugars after the treatment of the biomass. It is known to use an enzymatic hydrolysis in the bio-refinery processes.

### OBJECTIVE

The objective of the invention is to improve an enzymatic hydrolysis. Another objective is to provide a new method for carrying out the enzymatic hydrolysis. Another objective is to produce a liquid fraction and a solid fraction in connection with the enzymatic hydrolysis.

### SUMMARY

The method for the enzymatic hydrolysis is characterized by what is presented in claim 1.

The apparatus for the enzymatic hydrolysis is characterized by what is presented in claim 15.

The liquid fraction is characterized by what is presented in claim 21.

The solid fraction is characterized by what is presented in claim 22.

The use of the liquid fraction is characterized by what is presented in claim 23.

The use of the solid fraction is characterized by what is presented in claim 24.

### BRIEF DESCRIPTION OF THE DRAWINGS

The accompanying drawings, which are included to provide a further understanding of the invention and constitutes a part of this specification, illustrate some embodiments of the invention and together with the description help to explain the principles of the invention. In the drawings:

FIG. 1 is a flow chart illustration of a method according to one embodiment,

FIG. 2 is a flow chart illustration of a method according to another embodiment,

FIG. 3 shows results from one example carried out according to one method embodiment,

FIG. 4 shows results from one example carried out according to one method embodiment,

FIG. 5 shows results from one example carried out according to one method embodiment,

FIG. 6 shows results from one example carried out according to one method embodiment,

FIG. 7 shows results from one example carried out according to one method embodiment, and

FIG. 8 shows results from one example carried out according to one method embodiment.

### DETAILED DESCRIPTION

In a method for an enzymatic hydrolysis plant based raw material, preferably cellulose based material, is hydrolysed by means of enzymes. In the method the plant based raw material (1) is fed to the first enzymatic hydrolysis stage (2), and the plant based raw material (1) is hydrolysed in at least two enzymatic hydrolysis stages (2,4). A liquid fraction (5a, 5b) comprising carbohydrates is separated from a solid fraction (6a,6b) in a solid-liquid separation stage (7a,7b) after each enzymatic hydrolysis stage (2,4), and the solid fraction (6a) is supplied to the next enzymatic hydrolysis stage (4) in which the solid fraction is treated, and the solid fraction (6b) is recovered after the last solid-liquid separation stage (7b), such as final or finish solid-liquid separation stage. Preferably, the solid fraction (6a,6b) comprising solids and the liquid fraction (5a,5b) are supplied out from the solid-liquid separation stage (7a,7b).

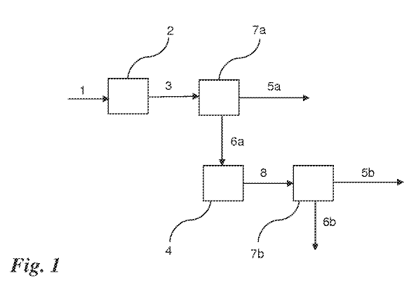

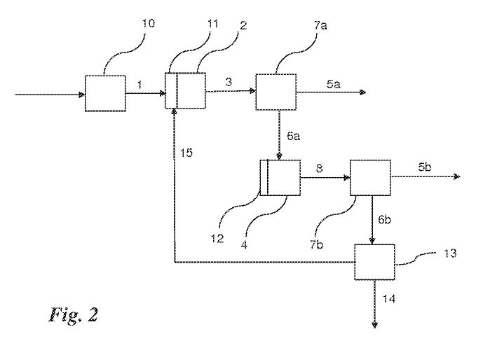

One embodiment of the method is shown in FIG. 1. Another embodiment of the method is shown in FIG. 2.

The apparatus comprises at least two enzymatic hydrolysis stages (2,4) in which the plant based raw material (1) is hydrolyzed, at least two solid-liquid separation stages (7a,7b) in which a liquid fraction (5a,5b) is separated from a solid fraction (6a,6b) after each enzymatic hydrolysis stage (2,4), and at least one feeding device for feeding the plant based raw material (1) to at least the first enzymatic hydrolysis stage (2). The enzymatic hydrolysis stage (4) after the first enzymatic hydrolysis stage (2) is arranged to treat the solid fraction (6a) separated in the solid-liquid separation stage (7a).

In one embodiment, the method and apparatus comprise two enzymatic hydrolysis stages. In one embodiment, the method and apparatus comprise more than two enzymatic hydrolysis stages.

The invention is based on an effective enzyuratic hydrolysis. In one process inhibitors, preferably inhibitors coming from cellulose based material, may be removed. According to an example, the inhibitor may belong to the group consisting of soluble lignin, organic acids, dissolved salts, glucose, xylose, oligomers, or other inhibitors or their combinations. Simultaneously, the recovery of the liquid fraction and solid fraction can be improved, and more pure solid fraction comprising lignin, can be formed.

In this context, an enzymatic hydrolysis means any enzymatic hydrolysis. In one embodiment, the enzymatic hydrolysis is an enzymatic hydrolysis of carbohydrates, e.g. cellulose.

In this context, a liquid fraction (5a,5b) means a liquid filtrate, which comprises mainly soluble carbohydrates and which is separated from the solid fraction. In a preferred embodiment, the liquid fraction includes carbohydrates, preferably C6 carbohydrates (C₆H₁₂O₆ or C₆ (H₂O)ₙ). Further, the liquid fraction may include C5 carbohydrates (C₅H₁₀O₅ or (C₅(H₂O)ₙ). The liquid fraction may comprise carbohydrates, such as monosaccharides (C₆H₁₂O₆ or C₅H₁₀O₅) disaccharides (C₁₂H₂₂O₁₁) oligosaccharides and/or polysaccharides ((C₆H₁₀O₅)ₙ or (C₅H₈O₄)ₙ). In one embodiment, the liquid fraction comprises soluble C5 and C6 carbohydrates and other carbohydrates. In one embodiment, the liquid fraction comprises soluble C5 carbohydrates and other carbohydrates. In one embodiment, the liquid fraction comprises soluble C6 carbohydrates and other carbohydrates. The liquid fraction may comprise also other components.

In this context, a solid fraction (6a,6b) means any solid fraction comprising solids, such as solid material, e.g. solid cake, high consistency slurry, agglomerates or the like, when a liquid fraction has been separated from the solid fraction. In a preferred embodiment, the solid fraction comprises lignin. Further, the solid fraction comprises carbohydrates, e.g. solid C6 carbohydrates (C₆H₁₂O₆ or C₆(H₂O)ₙ). The solid fraction may comprise also other carbohydrates and other components.

In this context, plant based raw material (1) means any plant based raw material, e.g. wood based raw material and/or other plant based material. Preferably, the plant based raw material is cellulose based material. The plant based raw material includes lignin, cellulose and hemicellulose. In one embodiment, the plant based raw material is formed from material selected from the group consisting of wood based material, wood, lignocellulosic biomass, agricultural residues, bagasse based material, sugarcane bagasse, corn based material, corn stover, wheat straw, rice straw, woody biomass, woody perennials, vascular plants and the like and their mixtures and their combinations. In one embodiment, the plant based raw material comprises wood based material or a mixture comprising wood based material. In one embodiment, the plant based raw material is wood based material or a mixture comprising wood based material. In one embodiment, the wood based material is selected from hardwood, softwood or their combination. In one embodiment, the plant based raw material comprises plant pieces, e.g. wood pieces.

In one embodiment, the plant based raw material (1) comprises carbohydrates and lignin. Preferably, the carbohydrates have Cₙ(H₂O)ₙ or Cₙ(H₂O)ₙ₋₁. The carbohydrates can comprise monosaccharides (C₆H₁₂O₆ or C₅H₁₀O₅), disaccharides (C₁₂H₂₂O₁₁), oligosaccharides and/or polysaccharides ((C₆H₁₀O₅)ₙ or (C₅H₈O₄)ₙ). Preferably, the plant based raw material comprises carbohydrates, such as soluble carbohydrates, e.g. C₅ carbohydrates (C₅H₁₀O₅ or C₅(H₂O)ₙ), and solid carbohydrates, e.g. C₆ carbohydrates (C₆H₁₂O₆ or C₆ (H₂O)ₙ).

The plant based raw material (1) may contain one or more material components. Preferably, the plant based raw material is in the form of suspension which contains liquid, such as water. Preferably, the plant based raw material is treated to dissolve hemicellulose.

In one embodiment, the plant based raw material (1) has been pre-treated, preferably by means of a suitable pretreatment. The pre-treatment stage (10) may be selected from the group consisting of physical pretreatment, such as milling, extrusion, microwave pretreatment, ultrasound pretreatment and freeze pretreatment, chemical pretreatment, such as acid pretreatment, alkaline pretreatment, ionic liquid pretreatment, organosolv pretreatment and ozonolysis, physico-chemical pretreatment, such as steam explosion pretreatment, ammonia fiber explosion pretreatment, CO₂ explosion pretreatment, liquid hot water pretreatment and wet oxidation, biological pretreatment and their combinations. In one embodiment, the plant based raw material is treated by the hydrolysis, e.g. acid hydrolysis, autohydrolysis, thermal hydrolysis, supercritical hydrolysis and/or subcritical hydrolysis, in which at least a part of lignin is separated from the raw material in connection with the hydrolysis. In one embodiment, the plant based raw material is treated by the steam explosion, in which hemicelluloses are treated and in which at least a part of polysaccharides of the hemicelluloses degrade into monosaccharides and oligosaccharides by means of a hydrolysis and in which pressure is rapidly released. In one embodiment, the plant based raw material is treated by the hydrolysis and by the steam explosion in one or more steps. In one embodiment, the plant based raw material is treated by the catalytic pretreatment, e.g. by using acid or base as catalyst.

In the pretreatment stage (10) the plant based raw material enters the reactor unit where the pretreatment takes place. The plant based raw material can be treated by means of one or more pretreatment. The treated plant based raw material (1) can be then supplied directly or via an intermediate step or via an intermediate storage to the enzymatic hydrolysis stage (2). Further, in one embodiment, the plant based raw material can be dewatered, e.g. by dewatering presses, and/or washed in one or two or more stages. The dewatering makes possible to separate sugar based streams.

In one embodiment, the plant based raw material (1) is diluted with liquid, preferably with water, e.g. fresh water or recirculated process water e.g. from a lignin purification process, or steam to form the feed to the first enzymatic hydrolysis stage (2). Preferably, the plant based raw material is diluted to suitable solid content. Dilution water may be added before the enzymatic hydrolysis stage, such as in a mixing stage or before the mixing stage. In one embodiment, feed concentration of the plant based raw material is 2-60% by weight (TS, total solids, at 105° C.), preferably 4-40% by weight (TS, total solids at 105° C.), more preferable 10-30% by weight (TS, total solids, at 105° C.), into the enzymatic hydrolysis stage.

In one embodiment, the plant based raw material (1) is fed by means of any suitable feeding device, such as a pump, e.g. a mono pump or piston pump or other suitable pump, into the enzymatic hydrolysis stage (2,4). Selection of the feeding device is based on e.g. feed concentration and/or viscosity of the plant based raw material.

In one embodiment, the enzymatic hydrolysis process is a continuous process. In one embodiment, the enzymatic hydrolysis process is a batch process. In one embodiment, the plant based raw material (1) is fed to the enzymatic hydrolysis stage (2) as a uniform flow. In one embodiment, the solid fraction (6a) is supplied to the next enzymatic hydrolysis stage (4) as a uniform flow. In one embodiment, the plant based raw material (1) is fed to the enzymatic hydrolysis stage (2) step by step or gradually for feeding material which have higher consistency than material in the enzymatic hydrolysis stage. In one embodiment, the solid fraction (6a) is supplied to the next enzymatic hydrolysis stage (4) step by step or gradually for feeding material which have higher consistency than material in the enzymatic hydrolysis stage.

In one embodiment, a residence time of the first enzymatic hydrolysis stage (2) is below 48 hours, in one embodiment below 36 hours, in one embodiment below 24 hours and in one embodiment below 12 hours. In one embodiment, residence time of the first enzymatic hydrolysis stage is over 2 hours, in one embodiment over 4 hours, in one embodiment over 6 hours and in one embodiment over 8 hours. In one embodiment, the residence time of the first enzymatic hydrolysis stage is between 2-48 hours, in one embodiment 4 36 hours, in one embodiment 6-24 hours and in one embodiment 8-12 hours.

In one embodiment, consistency of the plant based raw material (1) is below 40%, in one embodiment below 30% and in one embodiment below 25% TS (total solids, at 105° C.) in the first enzymatic hydrolysis stage (2). In one embodiment, the consistency of the plant based raw material is over 4%, in one embodiment over 10% and in one embodiment over 15%, TS (at 105° C.) in the first enzymatic hydrolysis stage. In one embodiment, the consistency of the plant based raw material is 4-40% TS (at 105° C.), in one embodiment 10-30% TS (at 105° C.) and in one embodiment 15-25% TS (at 105° C.), in the first enzymatic hydrolysis stage. In one embodiment, the consistency of the plant based raw material is 4-10% TS (at 105° C.) in the first enzymatic hydrolysis stage.

In one embodiment, the solid fraction (6a) is diluted with liquid, preferably with water, e.g. fresh water or recirculated process water e.g. from a lignin purification process, or steam in connection with the enzymatic hydrolysis stage and/or before the supplying to the next enzymatic hydrolysis (4). Preferably, the solid fraction is diluted to suitable solid content. Dilution water may be added before the enzymatic hydrolysis, such as in a mixing stage or before the mixing stage. In one embodiment, temperature of the second or any later enzymatic hydrolysis stage (4) is adjusted by means of temperature of the dilution liquid. In one embodiment, the solid fraction (6a) is supplied without the dilution to the next enzymatic hydrolysis (4).

In one embodiment, a residence time of the second or any later enzymatic hydrolysis stage (4) is below 72 hours, in one embodiment below 56 hours, in one embodiment below 50 hours, in one embodiment below 49 hours, in one embodiment below 48 hours and in one embodiment below 36 hours. In one embodiment, the residence time of the second or any later enzymatic hydrolysis stage is over 6 hours, in one embodiment over 12 hours, in one embodiment over 18 hours, in one embodiment over 20 hours, in one embodiment over 22 and in one embodiment over 24 hours. In one embodiment, the residence time of the second or any later enzymatic hydrolysis stage is 6-72 hours, in one embodiment 12-56 hours, in one embodiment 18-50 hours, in one embodiment 20-49 hours, in one embodiment 22-48 hours and in one embodiment 24-36 hours. In one embodiment, the residence time of the second enzymatic hydrolysis stage (4) is below 72 hours, in one embodiment below 56 hours, in one embodiment below 50 hours, in one embodiment below 49 hours, in one embodiment below 48 hours and in one embodiment below 36 hours. In one embodiment, the residence time of the second enzymatic hydrolysis stage is over 6 hours, in one embodiment over 12 hours, in one embodiment over 18 hours, in one embodiment over 20 hours, in one embodiment over 22 hours and in one embodiment over 24 hours. In one embodiment, the residence time of the second enzymatic hydrolysis stage is 6-72 hours.

In one embodiment, the residence time of the first enzymatic hydrolysis stage (2) is shorter than the residence time of the second or any later enzymatic hydrolysis stage (4). According to an example, the residence time of the first enzymatic hydrolysis stage (2) is 8-12 hours and the residence time of the second or any later enzymatic hydrolysis stage (4) is 24-48 hours.

In one embodiment, the total residence time of the first enzymatic hydrolysis stage (2) and the later enzymatic hydrolysis stages (4) is over 24 hours, in one embodiment over 36 hours, in one embodiment over 48 hours, in one embodiment over 56 hours, in one embodiment over 72 hours and in one embodiment over 80 hours.

In one embodiment, the method, the apparatus or the process comprises at least three enzymatic hydrolysis stages in which the first enzymatic hydrolysis stage is short, the middle enzymatic hydrolysis stage or stages are longer and the last enzymatic hydrolysis stage is long. According to an example, the residence time of the first enzymatic hydrolysis stage is between 4-36 hours, in one embodiment 6-24 hours and in one embodiment 8-12 hours, and the residence time of the middle enzymatic hydrolysis stage or stages is between 6-72 hours, in one embodiment 12-56 hours, in one embodiment 18-50 hours, in one embodiment 22-48 hours and in one embodiment 24-36 hours, and the residence time of the last enzymatic hydrolysis stage is between 30-100 hours. In one embodiment, the residence time of the first enzymatic hydrolysis stage is shorter than the residence time of the middle enzymatic hydrolysis stage or stages, and the residence time of the last enzymatic hydrolysis stage is at least equally long as the residence time of the first enzymatic hydrolysis stage. In one embodiment, the residence time of the first enzymatic hydrolysis stage is shorter than the residence time of the middle enzymatic hydrolysis stage or stages, and the residence time of the last enzymatic hydrolysis stage is longer than the residence time of the first enzymatic hydrolysis stage. In one embodiment, the residence time of the first enzymatic hydrolysis stage is shorter than the residence time of the middle enzyuratic hydrolysis stage or stages, and the residence time of the last enzymatic hydrolysis stage is at the same level as the residence time of the first enzymatic hydrolysis stage, e.g. substantially equally long as the residence time of the first enzymatic hydrolysis stage. In one embodiment, the method or the process comprises at least three enzymatic hydrolysis stages in which the first enzymatic hydrolysis stage is short, the middle enzymatic hydrolysis stage or stages are longer and the last enzymatic hydrolysis stage is short. According to an example, the residence time of the first enzymatic hydrolysis stage is between 4-36 hours, in one embodiment 6-24 hours and in one embodiment 8-12 hours, and the residence time of the middle enzymatic hydrolysis stage or stages is between 6-72 hours, in one embodiment 12-56 hours, in one embodiment 18-50 hours, in one embodiment 22-48 hours and in one embodiment 24-36 hours, and the residence time of the last enzymatic hydrolysis stage is between 4-36 hours, in one embodiment 6 24 hours and in one embodiment 8-12 hours. In one embodiment, the residence time of at least the second enzymatic hydrolysis stage is longer than the residence time of the first enzymatic hydrolysis stage. In one embodiment, the last enzymatic hydrolysis stage is long, e.g. 30-100 hours. In one embodiment, the residence time of the last enzymatic hydrolysis stage depends on an amount of active enzyme in the last enzymatic hydrolysis stage. In one embodiment, the last enzymatic hydrolysis stage is performed without an enzyme addition. In one embodiment, an enzyme is added into the last enzymatic hydrolysis stage. In one embodiment, a purification of the solid fraction, e.g. lignin, is performed in the last enzymatic hydrolysis stage. In one embodiment, an amount of carbohydrates is below 15% by weight, preferably below 10% by weight, more preferably below 5% by weight, in a solid fraction (6b) after the last enzymatic hydrolysis stage.

In one enzymatic hydrolysis process, the residence time of the first enzymatic hydrolysis stage may be longer than the residence time of the second or any later enzymatic hydrolysis stage.

In one embodiment, consistency of the solid fraction (6a) is below 40%, in one embodiment below 30%, TS (total solids, at 105° C.) in the second or any later enzymatic hydrolysis stage (4). In one embodiment, the consistency of the solid fraction (6a) is over 10%, in one embodiment over 20%, TS (at 105° C.) in the second or any later enzymatic hydrolysis stage. In one embodiment, the consistency of the solid fraction (6a) is 10-40%, in one embodiment 20-30%, TS (at 105° C.) in the second or any later enzymatic hydrolysis stage. In one embodiment, the consistency of the solid fraction (6a) is below 40%, in one embodiment below 30%, TS (total solids, at 105° C.) in the second enzymatic hydrolysis stage (4). In one embodiment, the consistency of the solid fraction (6a) is over 10%, in one embodiment over 20%, TS (at 105° C.) in the second enzymatic hydrolysis stage. In one embodiment, the consistency of the solid fraction (6a) is 10-40%, in one embodiment 20-30%, TS (at 105° C.) in the second enzymatic hydrolysis stage.

In one embodiment, the consistency in the second or any later enzymatic hydrolysis stage (4) is higher than the consistency in the first enzymatic hydrolysis stage (2).

In one embodiment, the plant based raw material (1) is treated so that the solid fraction (6a) contains over 80% fine solid particles which are fiber-like or indefinable particles smaller than 0.2 mm, defined by an optical measurement device, e.g. by Metso FS5, before the second or any later enzymatic hydrolysis stage (4). In one embodiment, the solid fraction (6a) contains over 85%, in one embodiment over 90%, in one embodiment over 92%, and in one embodiment over 94%, fine solid particles which are fiber-like or indefinable particles smaller than 0.2 mm, defined by Metso FS5. In one embodiment, the plant based raw material (1) is treated so that the solid fraction (6a) comprises fine solid particles which have particle size Mode between 18-300 μm, defined by Coulter LS230, before the second or any later enzymatic hydrolysis stage (4). In one embodiment, the solid fraction (6a) comprises fine solid particles which have Particle size Mode 19-200 μm, in one embodiment 20-150 μm, in one embodiment 20-120 μm, and in one embodiment 21-75 μm, defined by Coulter LS230. In one embodiment, the plant based raw material (1) is treated so that the viscosity of the solid fraction (6a) is below 18000 mPas at 15% dry matter content, measured by Brookfield viscosity device at 45° C. with 10 rpm and spindel type “Vane”, before the second or any later enzymatic hydrolysis stage (4). In one embodiment, the viscosity of the solid fraction (6a) is below 18000 mPas, in one embodiment below 13000 mPas, in one embodiment below 10000 mPas, and in one embodiment below 8000 mPas, at 15% dry matter content, measured by Brookfield viscosity device at 45° C. with 10 rpm and spindel type “Vane”. The plant based raw material (1) can be pre-treated and/or particle size and viscosity of the solid fraction (6a) can be determined according to patent application PCT/F12016/050075 or PCT/F12016/050076.

In one embodiment, the method comprises at least one mixing stage (11,12) in connection with the enzymatic hydrolysis stage (2,4), e.g. before the enzymatic hydrolysis stage or in the enzymatic hydrolysis stage or during the enzymatic hydrolysis. In one embodiment, the method comprises the mixing stage in connection with the first enzymatic hydrolysis stage. In one embodiment, the method comprises the mixing stage in connection with the enzymatic hydrolysis stages following the first enzymatic hydrolysis stage, e.g. in connection with the second enzymatic hydrolysis stage or in connection with any enzymatic hydrolysis stage following the second enzymatic hydrolysis stage. In one embodiment, the method comprises the mixing stage in connection with any desired enzymatic hydrolysis stage. Preferably, the mixing is a mixing wherein there is sufficient shear force for mixing liquid and solids into a homogenous mixture during the mixing. Further, solids can be disintegrated by means of the effective mixing. Solid particles can break down leading to higher specific surface. In one embodiment, material temperature may be increased by 5-15° C. during the mixing stage. In one embodiment, the apparatus comprises at least one mixing device which may be selected from the group consisting of a mixer, screw mixer, pump, other suitable device or their combination.

In one embodiment, pH is adjusted before the enzymatic hydrolysis stage (2,4), e.g. in the mixing stage or before the mixing stage, or during the enzyuratic hydrolysis stage. In one embodiment, pH is between 3-8, in one embodiment between 3.5-7 and in one embodiment between 4-6. In one embodiment, pH is adjusted so that pH is favorable for the enzyme used in the process.

In one embodiment, dewatering is carried out after the first enzymatic hydrolysis stage (2).

Preferably, the method comprises the solid-liquid separation stage (7a,7b) after each enzymatic hydrolysis stage (2,4). In one embodiment, the apparatus comprises at least one solid-liquid separation device. In one embodiment, the apparatus comprises more than one solid-liquid separation device. In one embodiment, each solid-liquid separation stage (7a,7b) comprises at least one solid-liquid separation device. In one embodiment, the solid-liquid separation stage (7a,7b) comprises more than one solid-liquid separation device. In one embodiment, each solid-liquid separation stage (7a,7b) comprises one solid-liquid separation device. In one embodiment, the liquid fraction (5a,5b) is separated from the solid fraction (6a,6b) by means of one solid-liquid separation device in more than one solid-liquid separation stage (7a,7b). In one embodiment, one solid-liquid separation device can be used in one or more solid-liquid separation stage (7a,7b). In one embodiment, one solid-liquid separation device can be used in more than one solid-liquid separation stage (7a,7b). In one embodiment, the separation device comprises one or more separation step, e.g. separation segment.

The solid-liquid separation stage may comprise one or more separation steps. In one embodiment, the solid-liquid separation stage comprises different procedures which may be done in one or more separation steps. In one embodiment, the liquid fraction is separated in one step. Alternatively, the liquid fraction may be separated in more than one step. In one embodiment, the liquid fraction is separated in each separation step.

Preferably, the solid-liquid separation stage (7a,7b) comprises the separation of the liquid fraction (5a,5b) from the solids, such as the solid fraction (6a,6b). In one embodiment, the liquid fraction (5a,5b) is separated from the solid fraction (6a,6b) by means of filtration, centrifugal treatment or their combinations. In one embodiment, the filtration is carried out by pressure, underpressure or overpressure.

In one embodiment, the solid-liquid separation device is based on a countercurrent washing. In one embodiment, the solid-liquid separation device is selected from the group consisting of filtration device, vacuum filtration device, press filter, belt press, centrifugal device and their combinations. In one embodiment, the solid-liquid separation device is selected from the group consisting of pressure filtration device, vacuum filtration device, filtration device based on underpressure, filtration device based on overpressure, filter press, other suitable press, centrifugal device and their combinations. In one embodiment, the solid-liquid separation device is a pressure filtration device, vacuum filtration device, filtration device based on underpressure or filtration device based on overpressure. In one embodiment, the solid-liquid separation device is a belt press, twin wire press or centrifuge. Alternatively, the solid-liquid separation device can be another washing device in which low amount of washing water is used and washing is done in high dry matter content. Then good recovery can be achieved. Alternatively, the solid-liquid separation device may be any suitable separation device.

In one embodiment, the solid-liquid separation stage (7a,7b) comprises a filtration in which the liquid fraction (5a,5b) is separated in a liquid form and solid material is formed. Preferably, pressure is used in the filtration. In one embodiment, liquid is separated by a pressure difference, such as by means of vacuum or overpressure. In one embodiment, the solid-liquid separation stage comprises a washing in which a displacement washing is carried out with small amount clean water in order to remove majority of sugars, inhibitors and other soluble compounds from the solid fraction (6a,6b) and to provide high recovery of soluble compounds. Preferably, ratio of washing water to solid is below 6, preferably below 3 and more preferably below 1.5. In one embodiment, the solid-liquid separation stage (7a,7b) comprises the filtration and washing. Preferably, high concentration and recovery of soluble material in the liquid phase can be achieved with small amount of clean water. Further, the solid fraction with minor amount of soluble compounds, or the solid fraction which is substantially free of soluble compounds, or the soluble compound lean solid fraction, can be achieved.

In one embodiment, the liquid fraction (5a,5b) is separated by means of a pressure filtration. In one embodiment, the apparatus comprises at least one pressure filtration device as the solid-liquid separation device.

In the different solid-liquid separation stages the separation can be carried out by means of similar or different separation methods or separation devices.

In one embodiment, the apparatus comprises means for supplying the intermediate product (3,8) from the enzymatic hydrolysis stage (2,4) to the solid-liquid separation stage (7a,7b). In one embodiment, the means for supplying the intermediate product (3,8) is selected from the group consisting of conveyor, screw, belt, pump, pipe, tube, duct, conduit, channel, outlet, other suitable feeding device and their combinations.

In one embodiment, the apparatus comprises means for supplying the solid fraction (6a) to the next enzymatic hydrolysis stage (4). In one embodiment, the means for supplying the solid fraction is selected from the group consisting of conveyor, screw, belt, pump, pipe, tube, duct, conduit, channel, outlet, other suitable feeding device and their combinations.

In one embodiment, the enzymatic hydrolysis stage (2,4) comprises a reactor, vessel, container, other suitable device or their combination in which the enzymatic hydrolysis is carried out.

In one embodiment, the apparatus comprises means for recovering the solid fraction (6b) after the last solid-liquid separation stage (7b). In one embodiment, the means for recovering the solid fraction is selected from the group consisting of assembly, outlet, conveyor, screw, belt, pipe, tube, duct, discharge outlet, discharge valve, discharge channel, conduit, other suitable device and their combinations.

In one embodiment, the liquid fraction (5a,5b) is recovered after each solid-liquid separation stage (7a,7b). In one embodiment, the apparatus comprises means for recovering the liquid fraction (5a,5b) after each solid-liquid separation stage (7a,7b). In one embodiment, the means for recovering the liquid fraction is selected from the group consisting of assembly, outlet, pipe, tube, duct, discharge outlet, discharge valve, discharge channel, conduit, other suitable device and their combinations.

In one embodiment, the enzyme is added in the second or any later enzymatic hydrolysis stage (4). In one embodiment, the enzyme is added in connection with the enzymatic hydrolysis stage (4), such as before the enzymatic hydrolysis stage or during the enzymatic hydrolysis. In one embodiment, the enzyme is added in the mixing stage or before the mixing stage. In one embodiment, the apparatus comprises an addition device for adding the enzyme.

In one embodiment, the enzyme is not added in the second or any later enzymatic hydrolysis stage (4). In one embodiment, the second or any later enzymatic hydrolysis stage (4) is carried out without an enzyme addition. It has been surprisingly observed that the second or any later enzymatic hydrolysis can be initiated and the enzymatic hydrolysis proceeds without the enzyme addition. Further, it has been observed that the enzyme is going on the solid fraction and the enzyme of the previous enzymatic hydrolysis stage (2) can be supplied to the next enzymatic hydrolysis stage (4) together with the solid fraction. In one embodiment, the enzyme is selected so that the enzyme has adhesion ability to the solids. In one embodiment, recycled enzyme is activated during the mixing.

In one embodiment the liquid fraction (5a,5b) is formed by means of the method. In one embodiment, the liquid fraction (5a) comprises soluble C5 and C6 carbohydrates after the first enzymatic hydrolysis stage (2). In one embodiment, the liquid fraction (5b) comprises soluble C6 carbohydrates after the second or any later enzymatic hydrolysis stage (4). The liquid fraction (5b) may comprise also C5 carbohydrates, preferably below 20%, more preferably below 10%, the most preferably below 5%, by weight of the carbohydrates, after the second or any later enzymatic hydrolysis stage. Preferably, the liquid fraction (5a, 5b) can contain other monosaccharides, disaccharides, oligosaccharides and/or polysaccharides. In one embodiment, the liquid fraction (5a,5b) contains galactose, glucose, mannose, arabinose, xylose, glucuronic acid and galacturonic acid. Preferably, the liquid fraction (5a,5b) is in the form of solution.

In one embodiment, at least a part of the liquid fraction (5a) is recovered by supplying out from the first solid-liquid separation stage (7a). In one embodiment, at least 50%, preferably at least 60%, more preferably at least 70%, of the soluble carbohydrates is supplied out from the first solid-liquid separation stage.

In one embodiment, at least a part of the liquid fraction (5b) is recovered by supplying out from the second or any later solid-liquid separation stage (7b). In one embodiment, at least 50%, preferably at least 60%, more preferably at least 70%, of the soluble carbohydrates is supplied out from the second or any later solid-liquid separation stage. In one embodiment, the liquid fraction (5b) comprises C6 carbohydrates over 80% by weight, preferably over 90% by weight, the most preferably over 95% by weight, of the carbohydrates. Preferably, the liquid fraction (5b) is a glucose rich fraction. Then the liquid fraction (5b) is sufficient pure that it can be used as such, or it can be concentrated and utilized after the concentration.

The liquid fraction (5a,5b) may be used as component in manufacturing a final product. The liquid fraction (5a) from the first solid-liquid separation and the liquid fraction (5b) from the second or any later solid-liquid separation can be utilized separately, or they can be combined or mixed and utilized as a mixture. In one embodiment, the liquid fraction (5a,5b) is used as such. In one embodiment, the liquid fraction (5a,5b) is supplied to a further processing. In one embodiment, the liquid fraction (5a,5b) is purified. In one embodiment, the liquid fraction (5a,5b) is concentrated. In one embodiment, the monomerization of the liquid fraction (5a,5b) is made before the further processing. In one embodiment, the liquid fraction (5a,5b) is supplied to a fermentation process. In one embodiment, the liquid fraction (5a,5b) is used as a source material in the fermentation. In one embodiment, the liquid fraction (5a,5b) is supplied to a hydrolysis process. In one embodiment, the liquid fraction (5a,5b) is used as a source material in the hydrolysis, such as in the acid hydrolysis, enzymatic hydrolysis or the like. In one embodiment, the liquid fraction (5a,5b) is supplied to a chemical treatment process. In one embodiment, the liquid fraction (5a,5b) is used as a source material in the chemical treatment. In one embodiment, the liquid fraction (5a,5b) is supplied to a polymerization process. In one embodiment, the liquid fraction (5a,5b) is used as a source material in the polymerization process. In one embodiment, the liquid fraction (5a,5b) is supplied to a depolymerization process. In one embodiment, the liquid fraction (5a,5b) is used as a source material in the depolymerization process. In one embodiment, the liquid fraction (5a,5b) is supplied to a catalytic treatment process. In one embodiment, the liquid fraction (5a,5b) is used as a source material in the catalytic treatment. In one embodiment, the liquid fraction (5a,5b) is supplied to a degradation process. In one embodiment, the liquid fraction (5a,5b) is used as a source material in the degradation process. In one embodiment, the liquid fraction (5a,5b) is supplied to an enzymatic treatment. In one embodiment, the liquid fraction (5a,5b) is used as a source material in the enzymatic treatment. In one embodiment, the liquid fraction (5a,5b) is supplied to a manufacture of binder. In one embodiment, the liquid fraction (5a,5b) is used as a source material in the manufacture of binder. In one embodiment, the liquid fraction (5a,5b) is supplied to a manufacture of feed. In one embodiment, the liquid fraction (5a,5b) is used as a source material in the manufacture of feed. In one embodiment, the liquid fraction (5a,5b) is supplied to a manufacture of food. In one embodiment, the liquid fraction (5a,5b) is used as a source material in the manufacture of food. The liquid fraction (5a,5b) may be supplied directly to the fermentation, hydrolysis, chemical treatment, catalytic treatment, polymerization process, depolymerization process, degradation process, enzymatic treatment, manufacture of binder, manufacture of feed, manufacture of food or other suitable process or their combinations, or alternatively via a suitable treatment step or an additional step, e.g. additional concentration step or purification step, to the fermentation, hydrolysis, chemical treatment, catalytic treatment, polymerization process, depolymerization process, degradation process, enzymatic treatment, manufacture of binder, manufacture of feed, manufacture of food or other suitable process or their combinations.

Preferably, the solid fraction (6a,6b) comprising solids is formed by means of the method. In one embodiment, the solid fraction (6b) comprises lignin after the last solid-liquid separation stage (7b). In one embodiment, the solid fraction (6b) comprises lignin and solid carbohydrates, such as C6 carbohydrates, such as (C₆H₁₂O₆ or (C₆(H₂O)ₙ), and other solid carbohydrates after the last solid-liquid separation stage (7b). Further, the solid fraction (6b) may comprise some residual soluble material. In one embodiment, the solid fraction (6b) is in the form of a solid material. In one embodiment, dry matter content of the solid material is over 30% by weight, preferably over 40% by weight, more preferably over 50% by weight, after the last solid-liquid separation stage. In one embodiment, dry matter content of the solid material is 15-80% by weight, in one embodiment 20 70% by weight, in one embodiment 30-60% by weight and in one embodiment 40-60% by weight, after the last solid-liquid separation stage. In one embodiment, the solid fraction (6b) contains soluble compounds below 15%, preferably below 6%, more preferably below 3% by weight, after the solid-liquid separation stage. In one embodiment, an amount of carbohydrates is below 25% by weight, preferably below 10% by weight, more preferably below 5% by weight, in the solid fraction (6b).

In one embodiment, the solid fraction is supplied out after the latest solid-liquid separation stage (7b). In one embodiment, at least a part of the solid fraction is supplied out after any previous solid-liquid separation stage. In one embodiment, at least a part of the solid fraction is supplied out after the first solid-liquid separation stage (7a).

The solid fraction (6b) may be used as component in manufacturing a final product. In one embodiment, the solid fraction (6b) is used as such. In one embodiment, the solid fraction (6b) is supplied to a further processing. In one embodiment, the solid fraction (6b) is supplied to a lignin purification for forming purified lignin. In one embodiment, the solid fraction (6b) is supplied to a lignin separation for separating lignin from the solid fraction. In one embodiment, the solid fraction (6b) is supplied to a hydrolysis which may be selected from the group consisting of acid hydrolysis, enzymatic hydrolysis, supercritical hydrolysis and/or subcritical hydrolysis and their combinations, or to a polymerization process, a depolymerization process, a degradation process, a chemical treatment, a manufacture of a composite material, lignin composite, activated carbon, carbon fiber, binder material, polymers, resins, phenolic component, dispersion agent or absorbent material, a manufacture of feed or food, or a combustion process or other suitable process or their combinations. The solid fraction may be supplied directly to the hydrolysis, polymerization process, depolymerization process, degradation process, chemical treatment, manufacturing processes of said materials, combustion process or other suitable process, or alternatively via a suitable treatment step or an additional step, e.g. additional separation step, purification step or dewatering step, to the hydrolysis, polymerization process, depolymerization process, degradation process, chemical treatment, manufacturing processes of said materials, combustion process or other suitable process.

In one embodiment, lignin (14) is separated in a lignin separation stage (13) from the solid fraction (6b) after the last solid-liquid separation stage (7b). Preferably, lignin is purified in connection with the enzymatic hydrolysis stage (4), e.g. the last enzymatic hydrolysis stage, and/or the lignin separation stage (13). The enzymes are denatured in the lignin separation stage (13). In one embodiment, the apparatus comprises at least one lignin separation device or lignin purification device. The lignin can be utilized as such, e.g. as a component in the final product or in the combustion. Alternatively, the lignin can be supplied to a further processing.

In one embodiment, a part of the solid fraction (15) preferably comprising residual cellulose or residual carbohydrates of the solid fraction, without active enzymes, may be recirculated from the lignin separation stage (13) to any previous enzymatic hydrolysis stage (2,4), in one embodiment to the first enzymatic hydrolysis stage (2). In one embodiment, the apparatus comprises at least one recirculation device for circulating residual cellulose or residual carbohydrates of the solid fraction from the lignin separation stage to the enzymatic hydrolysis stage.

The method and the apparatus can be used for treating materials comprising inhibitors, and for manufacturing lignin, carbohydrates and chemicals, and for removing inhibitors. By means of the method and the apparatus the enzymatic hydrolysis can be improved, the enzyme dosage can be decreased, residence time or reaction time of the enzymatic hydrolysis can be shortened, consistency can be increased in the enzymatic hydrolysis, purity of lignin can be improved, and/or the conversion of carbohydrates can be improved.

The method and the apparatus provide the solid fraction and liquid fraction with good quality. The solid fraction has very high concentration of lignin. Further, the solid fraction has very high purity. When inhibitors are removed together with the liquid fraction in at least two steps, more purified solid fraction can be provided in the process. Further, raw material with inhibitors and undesired agents can be used as a source material in the process. Also the carbohydrate recovery and conversion can be improved. Further, the method and the apparatus decrease post-treating costs of the solid fraction and also liquid fraction.

The method and the apparatus provide an industrially applicable, simple and affordable way of carrying out the enzymatic hydrolysis. The method or the apparatus is easy and simple to realize as a production process. The method and the apparatus are suitable for use in the manufacture of the different lignin and sugar based fractions and final products from different starting materials.

### EXAMPLES

Some embodiments of the invention are described in more detail by the following examples with reference to accompanying drawings.

### Example 1

In this example, the enzymatic hydrolysis is carried out in two stages, and a solid fraction and liquid fraction are produced according to a process of FIG. 1.

The plant based raw material (1) is fed into the first enzymatic hydrolysis stage (2). The plant based raw material (1) may be diluted with liquid before the first enzymatic hydrolysis stage (2). After the first enzymatic hydrolysis stage (2), an intermediate product (3) of the enzymatic hydrolysis is supplied into a solid-liquid separation stage (7a) comprising a filtration device. A liquid fraction (5a) comprising soluble C5 and C6 carbohydrates is separated from the solids in the separation stage (7a). A solid fraction (6a) containing e.g. lignin, solid carbohydrates, some soluble sugar, oligomer and polymer residual is removed from the separation stage (7a).

The solid fraction (6a) is supplied to the next enzymatic hydrolysis stage (4). The solid fraction (6a) may be diluted with liquid before the next enzymatic hydrolysis stage (4). After the second enzymatic hydrolysis stage (4), an intermediate product (8) of the enzymatic hydrolysis is supplied into a solid-liquid separation stage (7b) comprising a filtration device. A liquid fraction (5b) comprising soluble C6 carbohydrates is separated from the solids in the separation stage (7b). A solid fraction (6b) containing e.g. lignin, some solid carbohydrates and some soluble carbohydrates is removed from the separation stage (7b) and is recovered after the last solid-liquid separation stage (7b).

### Example 2

In this example, the enzymatic hydrolysis is carried out in two stages, and a solid fraction and liquid fraction are produced according to a process of FIG. 2.

The plant based raw material (1) is fed into the first enzymatic hydrolysis stage (2). The plant based raw material has been treated by means of pretreatment (10), e.g. by physical, chemical or physic-chemical treatment such as by microwave or ultrasound treatment, or by steam explosion. The plant based raw material (1) may be diluted with liquid in a mixing stage (11) in connection with the enzymatic hydrolysis stage (2) before the first enzymatic hydrolysis.

After the first enzymatic hydrolysis stage (2), an intermediate product (3) of the enzymatic hydrolysis is supplied into a solid-liquid separation stage (7a) comprising a filtration device. A liquid fraction (5a) comprising soluble C5 and C6 carbohydrates is separated from the solids in the separation stage (7a). A solid fraction (6a) containing e.g. lignin, solid carbohydrates, some soluble sugar, oligomer and polymer residual is removed from the separation stage (7a).

The solid fraction (6a) is supplied to the next enzymatic hydrolysis stage (4). The solid fraction (6a) may be diluted with liquid in a second mixing stage (12) in connection with the enzymatic hydrolysis stage (4) before the second enzymatic hydrolysis. After the second enzymatic hydrolysis stage (4), an intermediate product (8) of the enzymatic hydrolysis is supplied into a solid-liquid separation stage (7b) comprising a filtration device. A liquid fraction (5b) comprising soluble C6 carbohydrates is separated from the solids in the separation stage (7b). A solid fraction (6b) containing e.g. lignin, some solid carbohydrates and some soluble carbohydrates is removed from the separation stage (7b) and is recovered after the last solid-liquid separation stage (7b).

Lignin (14) is separated from the solid fraction (6b) in a lignin separation stage (13) comprising a lignin separation device. The enzymes are denatured in the lignin separation stage (13). A part of the solid fraction (15) comprising residual cellulose and residual carbohydrates may be recirculated from the lignin separation stage (13) to the first enzymatic hydrolysis stage (2).

### Example 3

In this example, a two-step enzymatic hydrolysis was studied.

The two-step enzymatic hydrolysis process was simulated and compared to a traditional one-step enzyuratic hydrolysis process in laboratory scale tests. Dilute acid pretreated and steam exploded birch was used as a substrate in the test. Commercially available enzyme mixture A was used in the enzymatic hydrolysis. The substrate was diluted by using distilled water, and pH was adjusted to 5, temperature was 50° C., enzyme dosage was 4% (total solids, at 105° C.) and initial dry matter content (total solids, at 105° C.) 15% in the experiments. 50 ml tubes containing 20 g of the substrate slurry was put into a mixer, and the mixer was placed in an incubator.

Reference sample tubes were taken out from the incubator after 6, 12, 48 and 72 hours. Two-step samples were taken out after the 1st enzymatic hydrolysis step either after 6 or 12 hours. The tubes were put in a centrifuge, rotating speed 1000 rpm with 5 minutes running time. A solid-liquid separation was done by taking the liquid phase out from the tube. The residual solid content in the 50 ml tube was diluted back to 20 g total weight of slurry for the second enzymatic hydrolysis step. Samples of the second enzyuratic hydrolysis step were taken out from the incubator after one or two days. Sugar analysis was done using standard HPLC methods from the liquid phase.

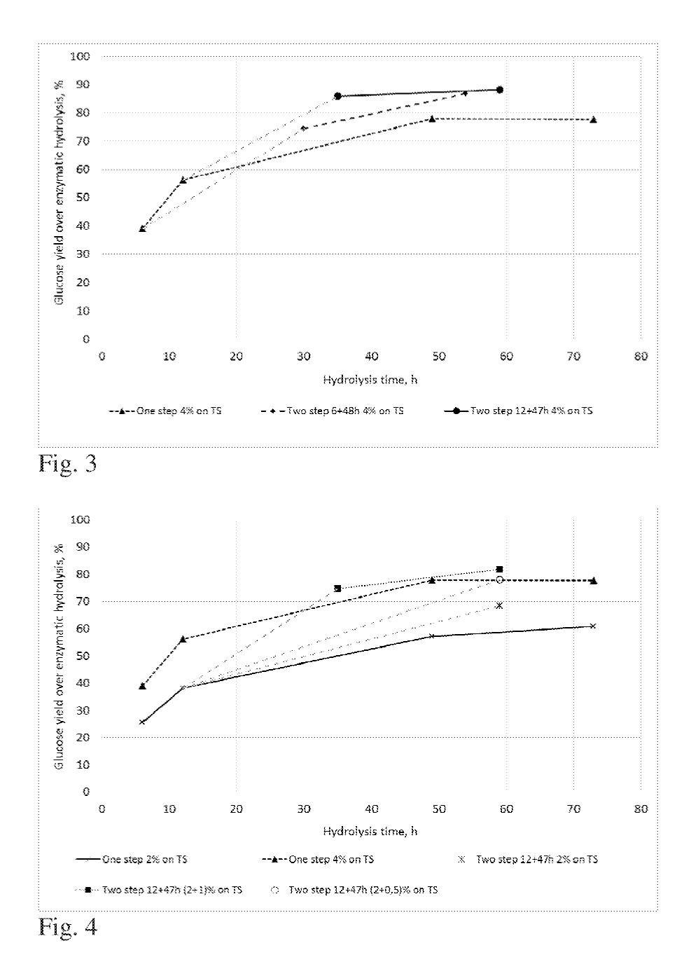

From FIG. 3 it can be seen that the two-step process will end up to 86% overall yield while the reference gave only 78% yield with the same enzyme dosage. Increase in yield with the two-step enzymatic hydrolysis process was 8%.

### Example 4

In this example, a two-step enzymatic hydrolysis was studied.

The two-step enzymatic hydrolysis process was simulated and compared to a traditional one-step enzymatic hydrolysis process in laboratory scale tests. Dilute acid pretreated and steam exploded birch was used as a substrate in the test. Commercially available enzyme mixture A was used in the enzymatic hydrolysis. The substrate was diluted by using distilled water, and pH was adjusted to 5, temperature was 50° C., and initial dry matter content (total solids, at 105° C.) 15% in the experiments. Enzyme dosages were 2% and 4% (total solids, at 105° C.) for the one-step process and 2% (total solids, at 105° C.) for the two-step process initially. 50 ml tubes containing 20 g of substrate slurry was put into a mixer, and the mixer was placed in an incubator.

Reference sample tubes were taken out from the incubator after 6, 12, 48 and 72 hours. Two-step samples were taken out after the 1st enzymatic hydrolysis step after 12 hours. The tubes were put in a centrifuge, rotating speed 1000 rpm with 5 minutes running time. A solid-liquid separation was done by taking the liquid phase out from the tube. The residual solid content in the 50 ml tube was diluted back to 20 g total weight of slurry for the second enzymatic hydrolysis step. In the two-step process there were also 0.5% and 1% (total solids, at 105° C.) enzyme addition into the second enzymatic hydrolysis step based on the original dry matter of the sample. Samples of the second enzymatic hydrolysis step were taken out from the incubator after one or two days. Sugar analysis was done using standard HPLC methods from the liquid phase.

From FIG. 4 it can be seen that the two-step process with 2% (total solids, at 105° C.) enzyme dosage will end up to 68% overall yield while the reference gave only 60% yield with the same enzyme dosage. Increase in yield with the two-step enzymatic hydrolysis process was 8%. 78% overall yield was achieved by adding 0.5% enzyme dosing (total solids, at 105° C.) into the second enzymatic hydrolysis step (2.5% totally). This is exactly same level as 4% dosing (total solids, at 105° C.) in the one-step process. Same yield was achieved by 1.5% less enzyme consumption if the two-step process was used. Over 80% overall yield was achieved by adding 1% enzyme dosing (total solids, at 105° C.) into the second enzymatic hydrolysis step (3% totally).

### Example 5

In this example, a two-step enzymatic hydrolysis was studied.

The two-step enzymatic hydrolysis process was simulated and compared to a traditional one-step enzymatic hydrolysis process in laboratory scale tests. Dilute acid pretreated and steam exploded birch was used as a substrate in the test. Commercially available enzyme mixture B was used in the enzymatic hydrolysis. The substrate was diluted by using tap water, and pH was adjusted to 4.5, temperature was 45° C., and initial dry matter content (total solids, at 105° C.) 15% in the experiments. Enzyme dosage was 6% (total solids, at 105° C.), and the first step was done in 10 litre reactor equipped with a mixing and heating system.

Slurry was dewatered to 40% dry matter content by a Büchner funnel after the first step except the one-step samples which were taken as such and put in 50 ml tubes, 20 g in each, into an incubator. Sugar analysis was done from the filtrates using standard HPLC methods. 1st enzymatic hydrolysis step was 16 hours. Dewatered solid material was diluted back to either 15% or 25% dry matter content and put in 50 ml tubes into the same incubator with one-step tubes for the second enzymatic hydrolysis step. Temperature in the incubator was adjusted to 45° C. and a windmill type of rotating tube mixer was used in the experiment. The tubes were put in a centrifuge after the enzymatic hydrolysis, rotating speed 1000 rpm with 5 minutes running time. A solid-liquid separation was done by taking the liquid phase out from the tube. Sugar analysis was done using standard HPLC methods from the liquid phase.

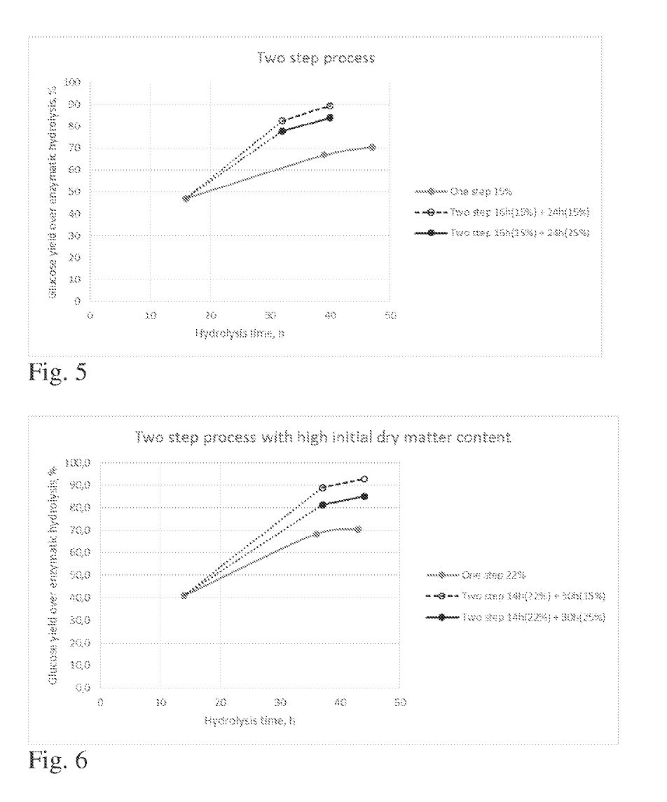

From FIG. 5 it can be seen that the two-step process with 6% enzyme dosage (total solids, at 105° C.) will end up to 84-88% overall yield while the reference gave only 70% yield with the same enzyme dosage. Increase in glucose yield with the two-step enzymatic hydrolysis process was over 14%.

### Example 6

In this example, a two-step enzymatic hydrolysis was studied.

The two-step enzymatic hydrolysis process was simulated and compared to a traditional one-step enzymatic hydrolysis process in laboratory scale tests. Dilute acid pretreated and steam exploded birch was used as a substrate in the test. Commercially available enzyme mixture B was used in the enzymatic hydrolysis. The substrate was diluted by using tap water, and pH was adjusted to 4.5, temperature was 45° C., and initial dry matter content (total solids, at 105° C.) 22% in the experiments. Enzyme dosage was 6% (total solids, at 105° C.), and the first step was done in 10 litre reactor equipped with a mixing and heating system.

Slurry was dewatered to 40% dry matter content by a Büchner funnel after the first step except the one-step samples which were taken as such and put in 50 ml tubes, 20 g in each, into an incubator. Sugar analysis was done from the filtrates by using standard HPLC methods. 1st enzymatic hydrolysis step was 14 hours. Dewatered solid material was diluted back to either 15% or 25% dry matter content and put in 50 ml tubes into the same incubator with one-step tubes for the second enzymatic hydrolysis step. Temperature in the incubator was adjusted to 45° C. and a windmill type of rotating tube mixer was used in the experiment. The tubes were put in a centrifuge after the enzymatic hydrolysis, rotating speed 1000 rpm with 5 minutes running time. A solid-liquid separation was done by taking the liquid phase out from the tube. Sugar analysis was done using standard HPLC methods from the liquid phase.

From FIG. 6 it can be seen that the two-step process with 6% enzyme dosage (total solids, at 105° C.) will end up to 84-92% overall yield while the reference gave only 70% yield with the same enzyme dosage. Increase in glucose yield with the two-step enzymatic hydrolysis process was over 14%.

### Example 7

In this example, a two-step enzymatic hydrolysis was studied.

The two-step enzymatic hydrolysis process was simulated and compared to a traditional one-step enzymatic hydrolysis process in laboratory scale tests. Dilute acid pretreated and steam exploded birch was used as a substrate in the test. The substrate contained about 98.7% fine solid particles which are fiber-like or indefinable particles smaller than 0.2 mm, defined by Metso FS5, and the substrate comprised fine solid particles which have particle size Mode 28.7 μm, defined by Coulter LS230. Commercially available enzyme mixture B was used in the enzymatic hydrolysis. The substrate was diluted by using tap water, and pH was adjusted to 4.5, temperature was 45° C., and initial dry matter content (total solids, at 105° C.) 15% in the experiments. Enzyme dosage was 6% (total solids, at 105° C.) and the first step was done in 10 litre reactor equipped with a mixing and heating system.

Slurry was dewatered to 40% dry matter content by a Büchner funnel after the first step except the one-step samples which were taken as such and put in 50 ml tubes, 20 g in each, into an incubator. Sugar analysis was done from the filtrates by using standard HPLC methods. 1st enzymatic hydrolysis step was 16 hours. Dewatered solid material was diluted back to 15% dry matter content and put in 50 ml tubes into the same incubator with one-step tubes for the second enzymatic hydrolysis step. The samples of the two-step process were mixed with gentle mixing and effective mixing before the second enzymatic hydrolysis step in the incubator. Temperature in the incubator was adjusted to 45° C. and a windmill type of rotating tube mixer was used in the experiment. The tubes were put in a centrifuge after the enzymatic hydrolysis, rotating speed 1000 rpm with 5 minutes running time. A solid-liquid separation was done by taking the liquid phase out from the tube. Sugar analysis was done using standard HPLC methods from the liquid phase.

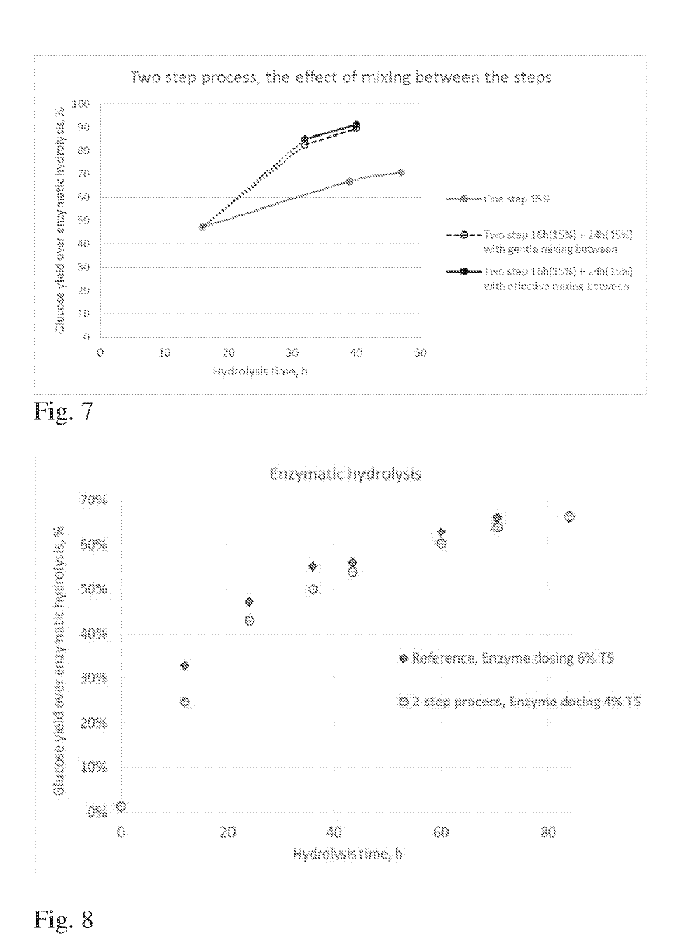

From FIG. 7 it can be seen that the two-step process with 6% enzyme dosage (total solids, at 105° C.) will end up to 90% overall yield while the reference gave only below 70% yield with the same enzyme dosage and the same hydrolysis time. Further, it can be seen that the yield of the two-step process was a little higher with the effective mixing between the enzymatic hydrolysis steps.

### Example 8

In this example, a two-step enzymatic hydrolysis was studied.

The two-step enzymatic hydrolysis process was simulated and compared to a traditional one-step enzyuratic hydrolysis process in laboratory scale tests. Dilute acid pretreated birch was used as a raw material in the test. Commercially available enzyme mixture B was used in the enzymatic hydrolysis. The raw material was diluted and pH was adjusted to 4.5, temperature was 45° C., and initial dry matter content (total solids, at 105° C.) 15% in the experiments. Enzyme dosage was 6% based on total solids (at 105° C.) of the raw material in the reference process and 4% based on total solids (at 105° C.) of the raw material in the two-step process.

In the two-step process, slurry was dewatered to 35% dry matter content (total solids, at 105° C.) by a vacuum filtration after the first step which was 12 hours. A solid fraction including enzymes was recovered and diluted with de-ionized water to target to the original total solids level. No pH adjustment was done and no new enzymes were added before the second step. The second step was up to 68 hours, and then combination was 84 hours. A big part of cellulose was hydrolyzed in the first step, and the rest of cellulose was hydrolyzed in the second step.

From FIG. 8 it can be seen that same sugar yield and sugar recovery can be achieved with ⅓ less of enzyme, when the two-step process is used.

The method and apparatus according to the present invention is suitable in different embodiments to be used in different enzymatic hydrolysis. Further, the method and apparatus according to the present invention is suitable in different embodiments to be used for producing the most different kinds of liquid and solid fractions from different raw materials.

The invention is not limited merely to the example referred to above; instead many variations are possible within the scope of the inventive idea defined by the claims.

1. A method for an enzymatic hydrolysis in which plant based raw material is hydrolysed by means of enzymes, the method comprising:
feeding the plant based raw material to the first enzymatic hydrolysis stage;
hydrolysing the plant based raw material in at least two enzymatic hydrolysis stages;
separating a liquid fraction comprising carbohydrates from a solid fraction in a solid-liquid separation stage after each enzymatic hydrolysis stage; and
supplying the solid fraction to the next enzymatic hydrolysis stage in which the solid fraction is treated, and the solid fraction is recovered after the last solid-liquid separation stage.

2. The method according to claim 1, wherein the residence time of the first enzymatic hydrolysis stage is 2-48 hours.

3. The method according to claim 1, wherein consistency of the plant based raw material is 4-40% in the first enzymatic hydrolysis stage.

4. The method according to claim 1, wherein the residence time of the second or any later enzymatic hydrolysis stage is 6-72 hours.

5. The method according to claim 1, wherein consistency of the solid fraction is 10-40% in the second or any later enzymatic hydrolysis stage.

6. The method according to claim 1, wherein the method comprises at least one mixing stage in connection with the enzymatic hydrolysis stage.

7. The method according to claim 1, wherein the plant based raw material or solid fraction is diluted with liquid before the enzymatic hydrolysis.

8. The method according to claim 1, wherein the liquid fraction is separated from the solid fraction by means of filtration, centrifugal treatment, or their combinations.

9. The method according to claim 1, wherein the liquid fraction is recovered after each solid-liquid separation stage.

10. The method according to claim 1, wherein the plant based raw material or solid fraction is fed to the enzymatic hydrolysis stage step by step or gradually.

11. The method according to claim 1, wherein the enzyme is added in the second or any later enzymatic hydrolysis stage.

12. The method according to claim 1, wherein the second or any later enzymatic hydrolysis stage is carried out without an enzyme addition.

13. The method according to claim 1, wherein lignin is separated in a lignin separation stage from the solid fraction after the last solid-liquid separation stage.

14. The method according to claim 1, wherein the plant based raw material is wood based material or a mixture comprising wood based material.

15. An apparatus for an enzymatic hydrolysis in which plant based raw material is hydrolysed by means of enzymes, wherein the apparatus comprises:
at least two enzymatic hydrolysis stages in which the plant based raw material is hydrolyzed;
at least one feeding device for feeding the plant based raw material to at least the first enzymatic hydrolysis stage; and
at least two solid-liquid separation stages in which a liquid fraction is separated from a solid fraction after each enzymatic hydrolysis stage; and
the enzymatic hydrolysis stage after the first enzymatic hydrolysis stage is arranged to treat the solid fraction separated in the solid-liquid separation stage.

16. The apparatus according to claim 15, wherein the apparatus comprises at least one solid-liquid separation device.

17. The apparatus according to claim 15, wherein the solid-liquid separation device is selected from the group consisting of filtration device, vacuum filtration device, press filter, belt press, centrifugal device, and their combinations.

18. The apparatus according to claim 15, wherein the apparatus comprises means for supplying the solid fraction to the next enzymatic hydrolysis stage.

19. The apparatus according to claim 15, wherein the apparatus comprises means for recovering the solid fraction after the last solid-liquid separation stage.

20. The apparatus according to claim 15, wherein the apparatus comprises means for recovering the liquid fraction after each solid-liquid separation stage.

21. A liquid fraction comprising carbohydrates formed by the method according to claim 1.

22. A solid fraction comprising lignin formed by the method according to claim 1.

23. A use of the liquid fraction obtainable by the method according to claim 1, wherein the liquid fraction is used as a source material in a fermentation, hydrolysis, chemical treatment, catalytic treatment, polymerization process, depolymerization process, degradation process, enzymatic treatment, manufacture of binder, manufacture of feed, manufacture of food, or other suitable process or their combinations.

24. A use of the solid fraction obtainable by the method according to claim 1, wherein the solid fraction is used as a source material in a hydrolysis, polymerization process, depolymerization process, degradation process, chemical treatment, manufacture of a composite material, lignin composite, activated carbon, carbon fiber, binder material, polymers, resins, phenolic component, dispersion agent or absorbent material, manufacture of feed, manufacture of food, combustion process, or other suitable process or their combinations.
